SEQUENCE LISTING (2 sequences)

SEQ ID NO: 1 - protein, 8 aa, Artificial Sequence
  Truncated Alpha Beta Peptide
KLVFFAED

SEQ ID NO: 2 - protein, 8 aa, Artificial Sequence
  Truncated Alpha Beta Peptide
KDEAFFVL

(19) United States
(12) Patent Application Publication
(10) Pub. No.: US 2023/0181770 A1
(43) Pub. Date: Jun. 15, 2023

(54) COMPOSITIONS AND METHODS OF TARGETING AND IMAGING AGED MICROGLIA WITH Aß PEPTIDE AMINO ACID RESIDUES FOR V-DOMAIN BINDING OF RAGE

(71) Applicant: Neurosn, Inc., Tucson, AZ (US)

(72) Inventors: Gilbert R. Gonzales, Tucson, AZ (US); Nigel R. Stevenson, Sugar Hill, GA (US); Jaime Simon, Angleton, TX (US)

(21) Appl. No.: 16/976,861

(22) PCT Filed: Mar. 1, 2019
(86) PCT No.: PCT/US2019/020339

§ 371 Date: Aug. 31, 2020

Related U.S. Application Data
(60) Provisional application No. 62/637,712, filed on Mar. 2, 2018.

(51) Int. Cl. A61K 51/08; A61K 51/04; A61K 9/00; A61P 25/28

(52) U.S. Cl. CPC A61K 51/088; A61K 51/0482; A61K 9/0019; A61P 25/28

(57) ABSTRACT

Low dose radiation, including conversion electron energy induces apoptosis in peripheral macrophages and CNS microglia. The transport of Sn-117m, a conversion electron emitter has been shown to be deliverable into the CNS across the blood-brain-barrier (BBB). The receptor for advanced glycation end products (RAGE) is a multi-ligand receptor member of the immunoglobulin super family which is able to bind A13 peptide and 13-sheet fibrils. It is expressed in endothelial cells, smooth muscle cells, microglia and neurons, and is implicated in the transport of A13 through the BBB, oxidative stress-mediated neurotoxicity, and adverse microglia inflammatory responses. The interaction between RAGE and its ligands is thought to result in pro-inflammatory gene activation. Enhanced levels of RAGE ligands in Alzheimer's disease are thought to contribute to the cause of this disorder. Embodiments of the invention use the RAGE multi-ligand site as an anchoring loci for a conversion electron emitting compound rather than as a receptor to intrinsically activate or block inflammation through the RAGE intracellular cascade through activation of the RAGE cytoplasmic tail (ctRAGE) and mammalian diaphanous 1 (DIAPH1).

### FIELD

The present invention is directed methods and compositions for targeting aged microglia in the central nervous system of a subject, and more particularly, to methods and compositions for imaging and inducing apoptosis in aged microglia with a radionuclide conjugate to treat neurodegenerative diseases.

### BACKGROUND

Alzheimer's disease (AD) produces a relentless decline of certain brain areas with resulting erosion of memory, the reduction the ability to perform tasks as well as affecting organizational abilities and creating poor judgment in the affected individual. The rate of progression from mild to moderate to severe AD can vary from person to person. The brain changes in AD can begin more than 20 years before the first symptoms appear. AD is associated with the presence of tangles and amyloid plaques. As tangles and amyloid plaques form in the brain, the areas of brain tissue that are affected become damaged and work less effectively. To date, most research has focused blocking or reducing amyloid plaque formation based on the amyloid cascade hypothesis. However, treatment for AD is still elusive despite the many compounds that have been tried in development and the billions of dollars expended to date.

Microglia are the resident phagocytes and innate immune cells of the brain. Investigators have recognized the importance of microglia in the homeostasis, as well as various pathologies, of the central nervous system. It is now widely accepted that clustered populations of reactive microglia are hallmarks of neurological disorders where neuro-inflammation is present and contributes to the mechanisms of neuronal damage. Subsets of reactive microglia called aged microglia are at least partially responsible for the cascade of events that lead to the deposition of amyloid beta (Aβ), a substance that is toxic to neurons as it accumulates in amyloid plaques. These microglia have been shown to be associated with amyloid plaques and neurons containing tau pathology, hallmarks of neurodegenerative diseases like AD. Accumulation of hyper-phosphorylated tau in neurons is correlated with progressive cognitive dysfunction and neuronal loss. Furthermore, reactive microglia have been associated with a variety of neurodegenerative diseases including AD, Parkinson's disease (PD) and amyotrophic lateral sclerosis (ALS).

The radionuclide Sn-117m's conversion electrons are a unique therapeutic particle emission with an average energy of 0.14 MeV that interact with tissue. Unlike beta rays that are emitted by most therapeutic radioisotopes, the Sn-117m conversion electrons are mono-energetic and travel an absolute maximum distance in tissue of ˜300 μm. This limits the therapeutic effect to tissue targeted by Sn-117m without damaging adjacent healthy tissue. Additionally, it has been demonstrated that at very low radiation doses, well below conventional DNA-breaking doses, the conversion electrons have a positive therapeutic effect by inducing apoptosis in macrophages and microglia. Sn-117m's unique conversion electron energy has several distinct advantages over traditional radiation therapy including an ideal two-week half-life and an unprecedented safety profile which allows shipping with no special handling procedures. Sn-117m also emits gamma photons, which are similar in energy to that of ^{99m}Tc, that can be imaged with a standard y-camera or single-photon emission computed tomography (“SPECT”). The gamma photons thus allow tissue targeted by Sn-117m to be imaged.

SUMMARY

Unlike the failed approach to treating AD through the “amyloid cascade hypothesis”, embodiments of the present invention are directed to methods of treating AD through the induction of apoptosis (programmed cell death) in aged microglia of the brain. This therapeutic approach is based on the “neuro-inflammatory hypothesis” as well as the “microglial dysfunction hypothesis”. These hypotheses hold that microglia that reside in the brain are at least partially responsible for the cascade of events that lead to the deposition of amyloid beta (AB), a substance that is toxic to the brain as it accumulates and is responsible for neuronal cell death. Microglia have also been shown to be associated with intra-neuronal tau pathology in the brain and tau is correlated with progressive cognitive dysfunction and neuronal loss. Embodiments of the present invention utilize radionuclide conjugates having a unique therapeutic energy form called conversion electrons (CE) that has been shown in experimental models to induce apoptosis in radiation doses that are extremely low. These CE doses are below typical therapeutic radiation DNA fracturing doses that lead to necrotic cell death such as used in cancer treatments. A CE compound has already been successfully delivered intravenously into the brain in humans, although not for the purposes of targeting aged microglia to treat neurodegenerative diseases like AD, PD, and ALS.

### DETAILED DESCRIPTION

Embodiments of the invention are directed to methods of inducing apoptosis of, imaging, or both inducing apoptosis of and imaging aged microglia in the central nervous system of a subject. The methods may treat neurodegenerative diseases in which aged microglia play a role in the underlying disease process by disrupting the neuro-inflammatory cascade that is implicated in these disorders. Exemplary neurodegenerative diseases that may be treated include AD, PD, and ALS. As used herein, “aged microglia” means microglia that express RAGE receptors at a higher level than regular microglia.

The methods utilize a radionuclide conjugate that includes a radionuclide and a targeting agent capable of binding RAGE receptors on aged microglia. Further embodiments of the invention are directed to methods of producing a radionuclide conjugate that is capable of targeting RAGE receptors on aged microglia. Additional embodiments of the invention are directed to compositions that include radionuclide conjugates that are capable of targeting RAGE receptors on aged microglia. The radionuclide conjugate is capable binding to the RAGE receptor and results in inhibition of aged microglia, which is believed to occur without internal cellular triggering of the cytoplasmic tail of RAGE and the intracellular effector, diaphanous-1.

The radionuclide is an isotope capable of producing a conversion election (CE). In an exemplary embodiment, the radionuclide is Sn-117m. In an embodiment, Sn-117m has at least a medium specific activity, i.e., an activity of at least 100 Ci/g. In another embodiment, the Sn-117m has a specific activity that is at least a high specific activity, i.e., an activity of at least 1,000 Ci/g. In yet another embodiment, the Sn-117m has a specific activity that is a very high specific activity, i.e., a specific activity of at least 10,000 Ci/g. For purposes of the present invention, the range for the specific activity of Sn-117m are defined as follows:
Low Specific Activity Sn-117m: <100 Ci/g; such as manufactured with reactors;
Medium Specific Activity Sn-117m: 100-1,000 Ci/g; such as manufactured with proton accelerators but low yield;
High Specific Activity Sn-117m: 1,000-10,000 Ci/g; such as manufactured with proton accelerators (but higher Sn-113 so limited use); primarily manufactured with alpha accelerators; and
Very High Specific Activity Sn-117m: >10,000 Ci/g; such as manufactured with alpha accelerators

The radionuclide is conjugated with a targeting agent. In embodiments of the invention, the targeting agent is a truncated version of the Aβ peptide amino acid chain, which has been identified as critical in binding to RAGE receptors expressed by aged microglia. The RAGE receptor is also found at the blood brain barrier and is responsible for active transport of Aβ from blood circulation to the brain. In embodiments of the invention, the targeting agent is an 8-amino acid chain Aβ(16-23) having the amino acid sequence KLVFFAED (SEQ ID NO. 1) or an amino acid chain Aβ(23-17) (modified with a terminal lysine (K)) having amino acid sequence KDEAFFVL (SEQ ID NO. 2). The amino acid chain Aβ(23-17) (modified with a terminal lysine (K)) having amino acid sequence KDEAFFVL (SEQ ID NO. 2) may also be referred to herein as K-Aβ(23-17). In embodiments of the invention, the radionuclide is conjugated to a mixture of targeting agents selected from Aβ(16-23) having the amino acid sequence KLVFFAED (SEQ ID NO. 1) and Aβ(23-17) having amino acid sequence KDEAFFVL (SEQ ID NO. 2). In embodiments of the invention may include a charge neutralizing blocking molecule at either end of the targeting agent, such as at the one or both ends of the Aβ fragments identified above. Further, longer versions of these Aβ fragments may be used to decrease steric hinderance that may be caused by the radionuclide/chelating agent.

In embodiments of the invention, the radionuclide is conjugated to the targeting agent with a chelating molecule, such as aminobenzyl DOTA (ABD), which the amine group on ABD is converted to an isothiocyanate group to form isothiocyanatebenzyl DOTA (IBD), or diethylene triamine pentaacetic acid (DTPA). Other chelants may be used.

In embodiments of the invention, the radionuclide conjugate includes a linking moiety between the chelating agent and the targeting agent. The linking moiety may function to allow a binding site between the chelating agent and the targeting agent. The linking moiety may also provide relief from steric hinderance that the chelating agent my cause with respect to the binding of the targeting agent and the RAGE receptor. In embodiments of the invention, the linking moiety may include an alkyl group containing chain having between 2 and 10 carbons, an ether group containing chain having between 1 and 10 ether groups, or other chains of subunits capable of relieving steric hinderance between the chelating agent and the binding of the targeting agent with the RAGE receptor. It will be appreciated that the alkyl group containing chain and ether group containing chain may include other components, such as alkyl rings, aromatic rings, amide groups, amino groups, hydroxyl groups, etc.

The radionuclide conjugate of Sn-117m with the above identified targeting agents (i.e., Aβ(16-23) KLVFFAED (SEQ ID NO. 1) or K-Aβ(23-17) KDEAFFVL (SEQ ID NO. 2)) can cross the intact or damaged blood brain barrier bind to RAGE receptors on aged microglia to induce apoptosis in the aged microglia via conversion electrons, thereby reducing the deposition of Aβ, and suppressing the propagation of hyper-phosphorylated tau protein in the brain. Through this mechanism, the presently described radionuclide conjugates can treat neurodegenerative diseases associated with aged microglia activity such as AD, PD, and ALS. In a preferred embodiment, the presently described radionuclide conjugates are administered to patients to treat AD. Furthermore, gamma emissions from Sn-117m can be imaged using a standard gamma camera or SPECT to assist with diagnosing conditions related to aged microglia, which may also be referred to herein as hyperactive microglia. In a preferred embodiment, the presently described radionuclide conjugates are administered to patients to image hyperactive microglia and diagnose, or support the diagnosis of, neurodegenerative diseases and, in particular, AD.

The structure of the exemplary Sn-117m-IBD-Aβ(16-23) KLVFFAED (SEQ ID NO. 1) conjugate is shown below.

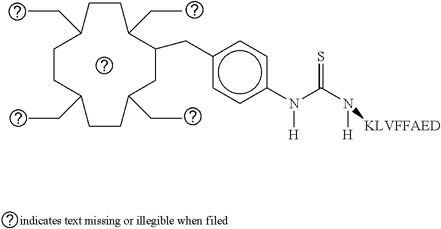

The structure of the exemplary Sn-117m-IBD-K-Aβ(23-17) KDEAFFVL (SEQ ID NO. 2) conjugate is shown below.

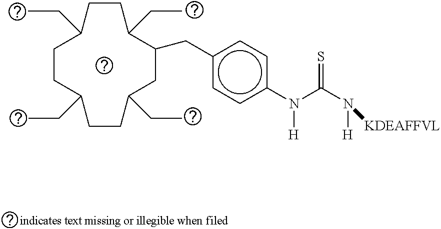

The structure of a variant of the exemplary Sn-117m-IBD-C-Aβ(16-23) CKLVFFAED (SEQ ID NO. 1) conjugate with an alternative Click chemistry linker groups is shown below.

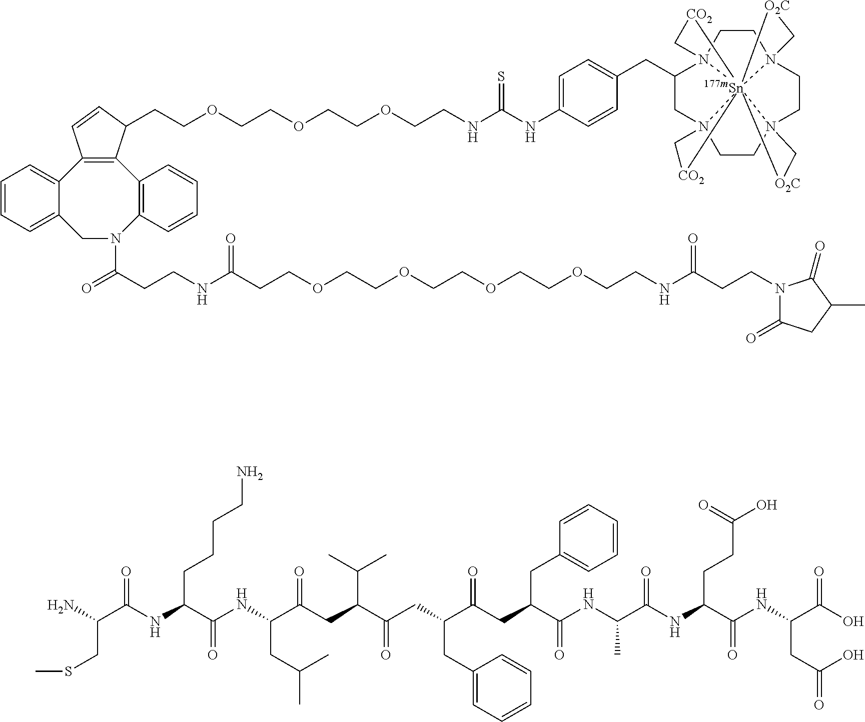

Embodiments of the radionuclide conjugate can be constructed and labeled with Sn-117m as follows, (1) Sn-117m first is attached to a bifunctional chelator such as aminobenzyl DOTA (ABD), and (2) the amine group on ABD is converted to an isothiocyanate group (IBD) and this is then conjugated to a lysine on the Aβ(16-23) to form Sn-IBD-Aβ(16-23) shown above. A similar procedure is followed for the formation of Sn-IBD-K-Aβ(23-17) also as shown above.

Embodiments of the radionuclide conjugate can be constructed and labeled with Sn-117m using Click chemistry as shown below. For this process, a terminal cysteine is added to the amino acid sequence of the targeting agent, which is coupled to DBCO-linked maleimide. The radionuclide Sn-117m is chelated to the chelating agent, which is linked to the amino acid sequence at the functional groups via the click reaction, as shown below to result in Sn-117m-IBD-C-Aβ(16-23) CKLVFFAED (SEQ ID NO. 1), as shown above. Similar reactions may used with SEQ ID NO. 2, which may be K and C modified.

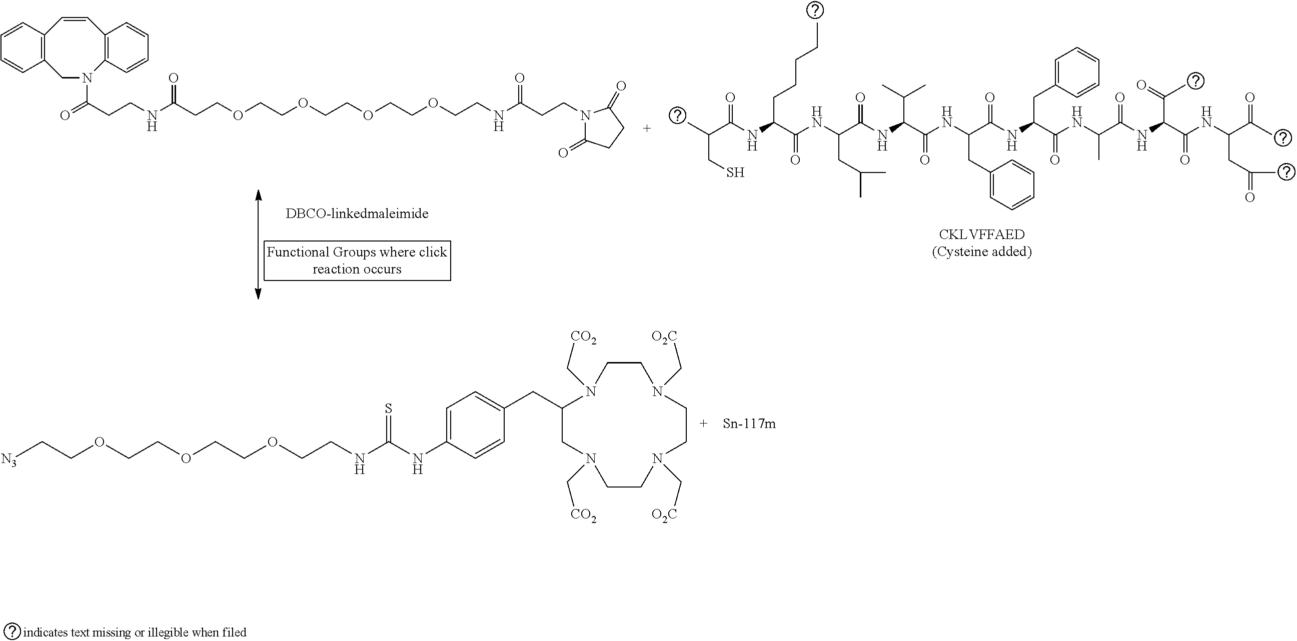

Embodiments of the radionuclide conjugate are capable of crossing the blood brain barrier into the CNS to target hyperactive ('aged') microglia when given peripherally. In embodiments, the radionuclide conjugate is administered intravenously. In other embodiments, radionuclide conjugate is delivered intra-arterially, such as into the carotid artery, which allows for the use of higher concentrations (i.e., higher localizing dosage) and in lower volume when administered, as compared to the dose needed when administered intravenously.

In embodiments of the invention, the radionuclide conjugate is administered in an amount effective to image aged microglia, induce apoptosis in aged microglia, or both image and induce apoptosis in aged microglia, which can in turn, treat a neurodegenerative disease such as AD, PD, and ALS. In a preferred embodiment, the radionuclide conjugate is injected at a dose sufficient to treat AD.

The amount effective to treat a neurodegenerative disease caused, at least in part, by aged microglia, is an amount that delivers a sufficient dose of the radionuclide conjugate to the central nervous system to result in a hormetic response in the central nervous system. The hormetic response may include inducing apoptosis in aged microglia, without inducing wider spread necrosis of tissue in the central nervous system. In embodiments, the amount administered is effective to image aged microglia in the subject. In further embodiments, the amount administered is effective to both induce apoptosis and image aged microglia in the subject. In embodiments, imaging can be conducted with a gamma camera or with single-photon emission computerized tomography (“SPECT”). In embodiments of the invention, the effective dose is below a dose that result in DNA, RNA, and polymerase fracturing. The amount administered can vary depending on the severity of the neurodegenerative disease, the route of administration, and the specific activity of the radionuclide. In an embodiment, the amount administered is sufficient to deliver a dose in a range from 100 μCi to 100 mCi to the central nervous system. In another embodiment, the amount administered is sufficient to deliver to the central nervous system a dose in a range from 500 μCi to 10 mCi, or a dose in a range from 3 mCi to 100 mCi. Lower doses, such as from 500 μCi to 10 mCi, may be administered via arterial injection, such as into the carotid artery. Higher dosages, such as 3 mCi to 100 mCi may be administered via intravenous injection.

### EXAMPLE 1

Animal studies—Truncated versions of the Aβ peptide amino acid chain has been identified as critical in binding to RAGE receptors. These 8 amino acid chains have demonstrated critical binding affinity. Attaching Sn-117m to these amino acids allows for targeting aged microglia within the radioisotope for subsequent therapeutic action.

Four APPSWE Model 2789 mice (AD) and 4 C57BL/6 mice (normal) receive an injection of 50 μCi of Sn-IBD-Aβ(16-23) per tail vein at day 0.

Additionally, four APPSWE Model 2789 mice (AD) and 4 C57BL/6 mice (normal) receive an injection of 50 μCi of Sn-IBD-K-Aβ(23-17) per tail vein at day 0.

All the animals are sacrificed on day 3 post Sn-Aβ-AA injection and brains preserved and autoradiography (AR) binding localization in AD-specific areas mapped.

Findings—Histopathological comparison and autoradiography dosimetry are measured in each mouse brain in comparative anatomic regions. Normal mouse brain AR distribution of RAGE binding should be minimal in cortical and hippocampal areas whereas the RAGE expressing mice will show an increase in RAGE binding with both the Sn-IBD-Aβ(16-23) and the Sn-IBD-K-Aβ(23-17).

### EXAMPLE 2

Four APPSWE Model 2789 mice (AD) and 4 C57BL/6 mice (normal) receive an injection of 50 μCi of Sn-IBD-Aβ(16-23) or 50 μCi of Sn-IBD-K-Aβ(23-17) per tail vein at day 0. The animals are sacrificed on day 3 post injection and brains preserved and autoradiography (AR) binding localization in AD-specific areas mapped. A comparison of binding localization between the two molecules is performed to verify binding of the radionuclide conjugate in the brains of the AD mice.

Systemically injected target RAGE expressed on the plasma membrane of aged microglia, irradiating them with conversion electrons, disrupting the neuro-inflammatory cascade that is implicated in several neurological disorders. The resulting radionuclide conjugate is capable of binding to the cellular receptor against RAGE that results in inhibition of aged microglia and is believed to occur without the internal cellular triggering of the cytoplasmic tail of RAGE and the intracellular effector, diaphanous-1.

In embodiments of the invention, the irradiation of microglia may be conducted using other radioisotopes including beta energy emitters to the cellular receptor against glycation end-products to result in inhibition of aged microglia and occurs without the internal cellular triggering of the cytoplasmic tail of RAGE and the intracellular effector, diaphanous-1.

While specific embodiments have been described in considerable detail to illustrate the present invention, the description is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

1. A method of inducing apoptosis, imaging, or both inducing apoptosis and imaging aged microglia in the central nervous system of a subject comprising administering to the subject a radionuclide conjugated to a targeting agent “radionuclide conjugate” in an amount effective to induce apoptosis, image, or both inducing apoptosis and imaging aged microglia in a subject.

2. The method of claim 1, wherein the radionuclide is Sn-117m.

3. The method of claim 2, wherein the Sn-117m has a specific activity that has at least a medium specific activity.

4. The method of claim 3, wherein the specific activity is at least 100 Ci/g.

5. The method of claim 3, wherein the specific activity is in a range from 100 Ci/g to 1000 Ci/g.

6. The method of claim of claim 2, wherein the Sn-117m has a specific activity that is at least a high specific activity.

7. The method of claim 6, wherein the specific activity is at least 1,000 Ci/g.

8. The method of claim 6 or 7, wherein the specific activity is in a range from 1,000 Ci/g to 10,000 Ci/g.

9. The method of claim 2, wherein the Sn-117m has a specific activity that is a very high specific activity.

10. The method of claim 9, wherein the specific activity is at least 10,000 Ci/g.

11. The method of claim 1, wherein the targeting agent is a truncated version of the amyloid β(Aβ) peptide amino acid chain capable of binding to the receptor for advanced glycation end products “RAGE” receptors on aged microglia.

12. The method of claim 1, wherein the targeting agent is an 8-amino acid chain Aβ(16-23) having the amino acid sequence KLVFFAED (SEQ ID NO. 1).

13. The method of claim 1, wherein the targeting agent is an 8-amino acid chain K-Aβ(23-17) having amino acid sequence KDEAFFVL (SEQ ID NO. 2).

14. The method of claim 1, wherein the targeting agent is selected from the group consisting of an 8-amino acid chain Aβ(16-23) having the amino acid sequence KLVFFAED (SEQ ID NO. 1), an 8-amino acid chain K-Aβ(23-17) having amino acid sequence KDEAFFVL (SEQ ID NO. 2), and combinations thereof.

15. The method of claim 1, wherein the radionuclide is conjugated to the targeting agent with a chelating agent.

16. The method of claim 15, wherein the radionuclide conjugate includes a linking moiety between the targeting agent and the chelating agent.

17. The method of claim 16, wherein the linking moiety is selected from an alkyl containing chain, an ether containing chain, or combinations thereof.

18. The method of claim 15, wherein the chelating agent is aminobenzyl DOTA (ABD), isothiocyanatebenzyl DOTA (IBD), diethylene triamine pentaacetic acid (DTPA), or combinations of ABD and DTPA.

19. The method of claim 1, wherein the radionuclide conjugate is Sn-117m-IBD-KLVFFAED (SEQ ID NO. 1).

20. The method of claim 1, wherein the radionuclide conjugate is Sn-117m-IBD-KDEAFFVL (SEQ ID NO. 2).

21. The method of claim 1, wherein the radionuclide conjugate is Sn-117m-DTPA-KLVFFAED (SEQ ID NO. 1).

22. The method of claim 1, wherein the radionuclide conjugate is Sn-117m-DTPA-KDEAFFVL (SEQ ID NO. 2).

23. The method of claim 1, wherein the radionuclide conjugate is selected from the group consisting of Sn-117m-IBD-Lₘ-KLVFFAED (SEQ ID NO. 1), Sn-117m-IBD-Lₘ-KDEAFFVL (SEQ ID NO. 2), Sn-117m-DTPA-Lₘ-KLVFFAED (SEQ ID NO. 1), Sn-117m-DTPA-KDEAFFVL (SEQ ID NO. 2), or combinations thereof, wherein Lₘ is a linker moiety.

24. The method of claim 23, wherein Lₘ is selected from an alkyl containing chain, an ether containing chain, or combinations thereof.

25. The method of claim 1, wherein the radionuclide conjugate is administered to the subject via systemic injection to the subject.

26. The method of claim 25 wherein the radionuclide conjugate is injected intra arterially to the subject.

27. The method of claim 26 wherein the radionuclide conjugate is injected into the carotid artery.

28. The method of claim 25 wherein the radionuclide conjugate is injected intravenously into the subject.

29. The method of claim 1 wherein the amount effective is an amount that delivers a sufficient dose of the radionuclide conjugate to the aged microglia to result in a hormetic response in the tissue.

30. The method of claim 29 wherein the amount effective is an amount that induces apoptosis in aged microglia in the central nervous system of the subject.

31. The method of claim 30 wherein the amount administered does not induce necrosis in the central nervous system of the subject.

32. The method of claim 1 wherein the amount administered is effective to image aged microglia in the central nervous system of the subject.

33. The method of claim 1 wherein the amount administered is effective to both induce apoptosis in aged microglia and image aged microglia in the central nervous system of the subject.

34. The method of claim 32 further comprising imaging aged microglia in the subject with a gamma camera, with single-photon emission computerized tomography (“SPECT”), or with combinations thereof.

35. The method of claim 1 wherein the amount administered is sufficient to deliver a dose to the central nervous system in a range from 100 μCi to 100 mCi.

36. The method of claim 1 wherein the amount administered is sufficient to deliver a dose to the central nervous system in a range from 500 μCi to 10 mCi.

37. The method of claim 1 wherein the amount administered is sufficient to deliver a dose to the central nervous system in a range from 3 mCi to 100 mCi.

38. The method of claim 1 wherein radionuclide conjugate further includes a homogeneous colloid of Sn-117m.

39. The method of claim 1 wherein the radionuclide conjugate further includes a non-homogenous colloid of Sn-117m.
